SEQUENCE LISTING (27 sequences)

SEQ ID NO: 1 - protein, 141 aa, Cotton leafroll dwarf virus
ASSETFVFSKDSLSGSSSGSITFGPSLSDCPAFSNGMLKAYHEYKISMVLLEFISEASSTSSGSISYEVDPHCKLSTLSSTINKFGITKNGRKQFAASFINGQEWHDTSEDQFRILYKGNGSSSIAGSFRVTIRCQFHNPK

SEQ ID NO: 2 - protein, 141 aa, Cotton leafroll dwarf virus
ASSETFVFSKDSLSGSSSGSITFGPSLSDCPAFSNGILKAYHEYKISMVLLEFISEASSTSSGSISYEVDPHCKLSTLSSTINKFGITKNGRKQFAASFINGQEWHDTSEDQFRILYKGNGSSSIAGSFKVTIRCQFHNPK

SEQ ID NO: 3 - protein, 229 aa, Cotton leafroll dwarf virus
PVPSRFWGYEGNPQCKILTAENDRNIDSRPLNFVSMYKWEDEKWDKVNLQAGYSRNDRRCMETYFVIPASRGKFHVYLEADGEFVVKHIGGDRDGNWLGNIAYDVSQRGWNIGDYKGCKISNYQSNTVFVAGHPDAEMNGKHFDAARAVEVDWFASFELTCDDEDGAWRIYPPPIQKDSSYNYTVSYGEYTEKYCEWGAVSVSIDEDNSTGTKSRIKPHKGAMMWSDPE

SEQ ID NO: 4 - protein, 126 aa, synthetic construct
MAQVQLQESGGGLVPPGGSLTLSCTASGFTLGYYAIGWFRQTPGNQRELVASYTSDGHINYKDSVKGRFTISRDGAKNTVWLQMNSLKPEDTAVYYCSFQQWTLVGDDAAQHTDYWGQGTQVTVSS

SEQ ID NO: 5 - protein, 9 aa, Synthetic construct
GFTLGYYAI

SEQ ID NO: 6 - protein, 14 aa, synthetic construct
ELVASYTSDGHINY

SEQ ID NO: 7 - protein, 18 aa, synthetic construct
SFQQWTLVGDDAAQHTDY

SEQ ID NO: 8 - protein, 117 aa, synthetic construct
MAQVQLQESGGGLVEAGGSLTLNCTASASIFRGNTMAWYRQAPGEQREFVASITTTGSRNYANSAYGRFTISNDNAKRSVYLHMNSLKPEDTGVYYCNKRFPPQGDWGQGTQVTVSS

SEQ ID NO: 9 - protein, 9 aa, synthetic construct
ASIFRGNTM

SEQ ID NO: 10 - protein, 14 aa, synthetic construct
EFVASITTTGSRNY

SEQ ID NO: 11 - protein, 9 aa, synthetic construct
NKRFPPQGD

SEQ ID NO: 12 - protein, 127 aa, synthetic construct
MAQVQLQESGGGLAQAGDSLRLSCAASGRTFNSFAMGWFRQAPGKEREFVAAIKWNGVTTDYADSMRGRFTISRDNAKNTMYMQMNTLKPEDTAIYYCAAKPTWATTNGRPTAYDYWGQGTQVTVSS

SEQ ID NO: 13 - protein, 9 aa, synthetic construct
GRTFNSFAM

SEQ ID NO: 14 - protein, 15 aa, synthetic construct
EFVAAIKWNGVTTDY

SEQ ID NO: 15 - protein, 18 aa, synthetic construct
AAKPTWATTNGRPTAYDY

SEQ ID NO: 16 - protein, 127 aa, synthetic construct
MAQVQLQESGGGLAQAGDSLRLSCAASGRTFNSFAMGWFRQAPGKEREFVAAINWNGVTTDYADSMKGRFTISRDNAKNTMYLQMNTLKPEDTAIYYCAAKPTWATTNGRPAAYDYWGQGTQVTVSS

SEQ ID NO: 17 - protein, 9 aa, synthetic construct
GRTFNSFAM

SEQ ID NO: 18 - protein, 15 aa, synthetic construct
EFVAAINWNGVTTDY

SEQ ID NO: 19 - protein, 18 aa, synthetic construct
AAKPTWATTNGRPAAYDY

SEQ ID NO: 20 - protein, 127 aa, synthetic construct
MAQVQLQESGGGLVQAGDSLRLSCAASGRTFNSFAMGWFRQAPGKEREFVAAIKWNGVTTDYADSMKGRFTISRDNAKNTMYLQMNTLKPEDTAIYYCAAKPTWATTNGRPTAYDYWGQGTQVTVSS

SEQ ID NO: 21 - protein, 9 aa, synthetic construct
GRTFNSFAM

SEQ ID NO: 22 - protein, 15 aa, synthetic construct
EFVAAIKWNGVTTDY

SEQ ID NO: 23 - protein, 18 aa, synthetic construct
AAKPTWATTNGRPTAYDY

SEQ ID NO: 24 - DNA, 18 bp, synthetic construct
  source 1..18: mol_type = other DNA
accctccaaggaacagag

SEQ ID NO: 25 - DNA, 21 bp, synthetic construct
  source 1..21: mol_type = other DNA
cgaataatctgatygggtcac

SEQ ID NO: 26 - DNA, 25 bp, synthetic construct
  source 1..25: mol_type = other DNA
aacgcgcccagtccgcacaaatacc

SEQ ID NO: 27 - DNA, 25 bp, synthetic construct
  source 1..25: mol_type = other DNA
accgggtttactggggattgcacgc

(19) United States
(12) Patent Application Publication
(10) Pub. No.: US 2025/0257122 A1
(43) Pub. Date: Aug. 14, 2025

(54) COTTON LEAFROLL DWARF VIRUS-BINDING ANTIBODIES AND USES THEREOF

(71) Applicants: The United States of America, as represented by the Secretary of Agriculture, Washington, DC (US); Cornell University, Ithaca, NY (US)

(72) Inventors: MICHELLE L. HECK, ITHACA, NY (US); JOSHUA S. CHAPPIE, ITHACA, NY (US); ALEJANDRO OLMEDO-VELARDE, ITHACA, NY (US); MYFANWY ADAMS, ITAHCA, NY (US)

(21) Appl. No.: 18/436,287

(22) Filed: Feb. 8, 2024

(51) Int. Cl. C07K 16/10; C12N 15/10; C12Q 1/6804; C12Q 1/6844; G01N 33/543; G01N 33/68

(52) U.S. Cl. CPC C07K 16/10; C12N 15/1096; C12Q 1/6804; C12Q 1/6844; G01N 33/543; G01N 33/6857; C07K 2317/565; C07K 2317/622; G01N 2333/08; G01N 2470/04

(57) ABSTRACT

The present disclosure relates to single-chain antibodies that specifically bind to the cotton leafroll dwarf virus (CLRDV), and methods for using such antibodies in the serological or molecular detection of CLRDV in plant or aphid samples. In particular, the disclosure relates to single-chain antibodies that recognize the CLRDV coat protein (CP) or the CLRDV n-terminal read-through domain (^{N}RTD).

### FIELD OF THE INVENTION

The disclosure relates to the field of agricultural biotechnology, in particular to single-chain antibodies that bind to the cotton leafroll dwarf virus (CLRDV), and use of such antibodies for detection of CLRDV in plant or aphid samples.

### SEQUENCE LISTING

The instant application contains a Sequence Listing XML required by 37 C.F.R. § 1.831 (a) which has been submitted in XML file format via the USPTO patent electronic filing system, and is hereby incorporated by reference in its entirety. The XML file was created on Feb. 8, 2024, is named 0019_23_Sequence_Listing, and has 25,252 bytes.

### BACKGROUND OF THE INVENTION

Cotton leafroll dwarf virus (CLRDV), an invasive Polerovirus member of the family Solemoviridae, is an emerging threat to cotton grown in the cotton belt region of the United States. The majority of poleroviruses studied to date are transmitted by specific species of phloem-feeding aphid vectors in a circulative manner that involves the movement of viruses across and within specific insect tissues. CLRDV is transmitted by the cotton aphid Aphis gossypii and possibly other vector species. The impact of this virus on cotton production is currently unknown, and management strategies are nonexistent. Companies like Agdia and Nano Diagnostics manufacture and sell antibodies for plant virus detection including those for some poleroviruses.

Although polyclonal and monoclonal antibodies have been produced in the development of enzyme-linked immunosorbent assay (ELISA) tests to detect the CLRDV in cotton and weeds, to date, however, no antibodies that recognize CLRDV are commercially available.

Thus, new antibodies that specifically recognize CLRDV are needed, as are serological and/or molecular detection assays designed for specifically detecting CLRDV in plant or aphid samples.

### SUMMARY OF THE INVENTION

Provided herein are single-chain antibodies that specifically recognize cotton leafroll dwarf virus (CLRDV), and the use of such single-chain antibodies to detect CLRDV in plant and/or aphid samples using serological or molecular detection assays.

In an embodiment, the disclosure relates to single-chain antibodies or fragments thereof that bind to CLRDV. In some embodiments of the disclosure, the single-chain antibody or antigen-binding fragment thereof recognizes CLRDV coat protein (CP) or CLRDV n-terminal read-through domain (^{N}RTD). In some embodiments of the disclosure, the single-chain antibodies or antigen-binding fragments thereof comprise a CDR1, CDR2, and CDR3; wherein, the CDR1 has a sequence as set forth in SEQ ID NO: 5; SEQ ID NO: 9; SEQ ID NO: 13; SEQ ID NO: 17; or SEQ ID NO: 21; the CDR2 has a sequence as set forth in SEQ ID NO: 6; SEQ ID NO: 10; SEQ ID NO: 14; SEQ ID NO: 18; or SEQ ID NO: 22; and the CDR3 has a sequence as set forth in SEQ ID NO: 7; SEQ ID NO: 11; SEQ ID NO: 15; SEQ ID NO: 19; or SEQ ID NO: 23.

In an embodiment, the disclosure relates to a method for determining the presence of CLRDV in a sample, the method comprising contacting the sample with at least one single-chain antibody or antigen-binding fragment thereof of claim 1 to produce an antibody/CLRDV complex; detecting the antibody/CLRDV complex; and determining that CLRDV is present when the antibody/CLRDV complex is detected.

In an embodiment, the disclosure relates to a method for determining the presence of CLRDV in a sample, the method comprising: contacting the sample with at least one single-chain antibody or antigen-binding fragment thereof of claim 1 to produce an antibody/CLRDV complex; releasing RNA from the antibody/CLRDV complex; using reverse transcriptase to prepare cDNA from the released RNA; amplifying the cDNA with CLRDV-specific primers; and determining that CLRDV is present when the CLRDV cDNA is amplified.

### BRIEF DESCRIPTION OF THE DRAWINGS

FIG. 1A and FIG. 1B provide pictorial representations of the CLRDV structural proteins and the designs of the individual CLRDV coat protein (CP) and N-terminal readthrough domain (^{N}RTD) constructs. FIG. 1A shows the CLRDV genome arrangement with the open reading frames (ORFs) numbered. ORF 3 encodes for the coat protein, CP; and ORF5 encodes the readthrough domain, RTD. Together ORF3 and ORF5 encode the viral readthrough protein (RTP). FIG. 1B shows a representation of the design of CLRDV CP and ^{N}RTD protein constructs. Dashed lines indicate the location of each construct relative to the rest of the RTP. ^{N}RTD indicates the N-terminal segment of the RTD and ^{C}RTD indicates the C-terminal segment of the RTD.

FIG. 2A and FIG. 2B depict images of SDS-PAGE gels of purified CLRDV CP and CLRDV ^{N}RTD constructs. FIG. 2A shows CLRDV CP (TX) and CLRDV CP (AL). FIG. 2B shows CLRDV ^{N}RTD (TX). The calculated molecular weight of each construct is shown below each gel.

FIG. 3 depicts a scheme of the preparation of camelid single-chain antibodies raised against the CLRDV structural proteins. Purified CLRDV CP (SEQ ID NO:1 and SEQ ID NO: 2) [10a] and ^{N}RTD (SEQ ID NO:3) [10b] proteins were used to immunize alpacas [12]. Peripheral blood lymphocytes were isolated and, after generation of a bacteriophage library and panning [16], CLRDV-specific antibodies were identified [18], from which the camelid single-chain antibodies B6 and E3 recognizing CP and A12, C12, and D12 recognizing the ^{N}RTD were isolated.

FIG. 4A and FIG. 4B depict SDS-PAGE gels showing the purified camelid single-chain antibodies. FIG. 4A shows B6 and E3 that recognize the CLRDV CP. FIG. 4B shows A12, D12, and C12 that recognize the CLRDV ^{N}RTD.

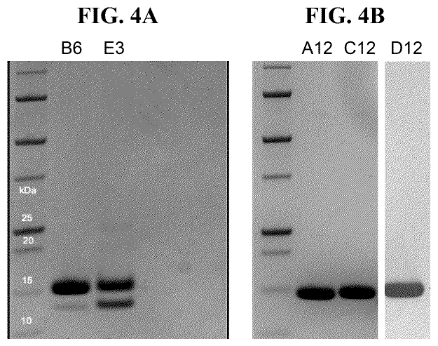

FIG. 5 depicts a diagram illustrating the four steps of the indirect ELISA assay to detect CLRDV virions using the camelid single-chain antibodies and a secondary anti-llama antibody conjugated with horseradish peroxidase (HRP) used herein.

FIG. 6A and FIG. 6B depict graphs of the change in OD at 650 nm using a mixture of the camelid single-chain antibodies in the indirect ELISA to detect CLRDV virions in different samples. FIG. 6A presents data for camelid single chain antibodies B6 and E3. FIG. 6B presents data for single chain antibodies A12, C12, and D12. The Y Axis shows the measured optical density. The X Axis shows the sample type: Infected plant; Healthy Plant; Blank consisting of PBS buffer only.

FIG. 7 depicts a schematic diagram of a double antibody sandwich (DAS) ELISA illustrating the four steps of the DAS-ELISA assay to detect CLRDV virions using the camelid single-chain antibodies and a secondary antibody/anti-PLRV conjugated with alkaline phosphatase (AP).

FIG. 8A and FIG. 8B depict graphs showing change in OD at 405 nm using a mixture of the camelid single-chain antibodies in a DAS-ELISA to detect CLRDV virions in different samples. FIG. 8A presents data for camelid single chain antibodies B6 and E3. FIG. 8B presents data for single chain antibodies A12, C12, and D12. The Y Axis shows the measured optical density at 405 nm. The X Axis shows the sample type: Infected plant; Healthy Plant; Blank consisting of PBS buffer only.

FIG. 9 depicts a schematic diagram for a molecular diagnostic approach for detection of CLRDV in aphid and cotton tissues using a mixture of camelid single-chain antibodies.

FIG. 10A and FIG. 10B depict agarose gels showing diagnostic data. FIG. 10A shows data for CLRDV CP. FIG. 10B shows data for ^{N}RTD. AR, samples from Arkansas, MS, samples from Mississippi, TN, samples from Tennessee; CLRDV-MS-2019 and Plasmid-CLRDV were positive controls; NTC (water non-template control), healthy seedling, and immunocapture (IC) blank were negative controls.

### BRIEF DESCRIPTION OF THE SEQUENCE LISTING

The amino acid and nucleotide sequences disclosed in the disclosure are listed in Table 1, below. Besides the sequence, the table includes the Sequence Identifier (SEQ ID) for each, the type, and a description of what each sequence is.

TABLE 1

| SEQ ID | Type | Description | Sequence |
|---|---|---|---|
| NO: 1 | AA | CLRDV TX coat | ASSETFVFSKDSLSGSSSGSITFGPSLSDCPA |
| | | protein | FSNGMLKAYHEYKISMVLLEFISEASSTSSG |
| | | | SISYEVDPHCKLSTLSSTINKFGITKNGRKQF |
| | | | AASFINGQEWHDTSEDQFRILYKGNGSSSIA |
| | | | GSFRVTIRCQFHNPK |
| NO: 2 | AA | CLRDV AL coat | ASSETFVFSKDSLSGSSSGSITFGPSLSDCPA |
| | | protein | FSNGILKAYHEYKISMVLLEFISEASSTSSGS |
| | | | ISYEVDPHCKLSTLSSTINKFGITKNGRKQF |
| | | | AASFINGQEWHDTSEDQFRILYKGNGSSSIA |
| | | | GSFKVTIRCQFHNPK |
| NO: 3 | AA | CLRDV TX | PVPSRFWGYEGNPQCKILTAENDRNIDSRP |
| | | NRTD protein | LNFVSMYKWEDEKWDKVNLQAGYSRNDR |
| | | | RCMETYFVIPASRGKFHVYLEADGEFVVK |
| | | | HIGGDRDGNWLGNIAYDVSQRGWNIGDYK |
| | | | GCKISNYQSNTVFVAGHPDAEMNGKHFDA |
| | | | ARAVEVDWFASFELTCDDEDGAWRIYPPPI |
| | | | QKDSSYNYTVSYGEYTEKYCEWGAVSVSI |
| | | | DEDNSTGTKSRIKPHKGAMMWSDPE |
| NO: 4 | AA | E3 single-chain | MAQVQLQESGGGLVPPGGSLTLSCTASGFTLGY |
| | | antibody | YAIGWFRQTPGNQRELVASYTSDGHINYKDSVK |
| | | sequence | GRFTISRDGAKNTVWLQMNSLKPEDTAVYYCSF |
| | | | QQWTLVGDDAAQHTDYWGQGTQVTVSS |
| NO: 5 | AA | E3 CDR1 | GFTLGYYAI |
| NO: 6 | AA | E3 CDR2 | ELVASYTSDGHINY |
| NO: 7 | AA | E3 CDR3 | SFQQWTLVGDDAAQHTDY |
| NO: 8 | AA | B6 single-chain | MAQVQLQESGGGLVEAGGSLTLNCTASASIFRG |
| | | antibody | NTMAWYRQAPGEQREFVASITTTGSRNYANSAY |
| | | sequence | GRFTISNDNAKRSVYLHMNSLKPEDTGVYYCNK |
| | | | RFPPQGDWGQGTQVTVSS |
| NO: 9 | AA | B6 CDR1 | ASIFRGNTM |
| NO: 10 | AA | B6 CDR2 | EFVASITTTGSRNY |
| NO: 11 | AA | B6 CDR3 | NKRFPPQGD |
| NO: 12 | AA | C12 single-chain | MAQVQLQESGGGLAQAGDSLRLSCAASGRTENS |
| | | antibody | FAMGWFRQAPGKEREFVAAIKWNGVTTDYADS |
| | | sequence | MRGRFTISRDNAKNTMYMQMNTLKPEDTAIYY |
| | | | CAAKPTWATTNGRPTAYDYWGQGTQVTVSS |
| NO: 13 | AA | C12 CDR1 | GRTFNSFAM |
| NO: 14 | AA | C12 CDR2 | EFVAAIKWNGVTTDY |
| NO: 15 | AA | C12 CDR3 | AAKPTWATTNGRPTAYDY |
| NO: 16 | AA | A12 single-chain | MAQVQLQESGGGLAQAGDSLRLSCAASGRTENS |
| | | antibody | FAMGWFRQAPGKEREFVAAINWNGVTTDYADS |
| | | sequence | MKGRFTISRDNAKNTMYLQMNTLKPEDTAIYYC |
| | | | AAKPTWATTNGRPAAYDYWGQGTQVTVSS |
| NO: 17 | AA | A12 CDR1 | GRTFNSFAM |
| NO: 18 | AA | A12 CDR2 | EFVAAINWNGVTTDY |
| NO: 19 | AA | A12 CDR3 | AAKPTWATTNGRPAAYDY |
| NO: 20 | AA | D12 single-chain | MAQVQLQESGGGLVQAGDSLRLSCAASGRTENS |
| | | antibody | FAMGWFRQAPGKEREFVAAIKWNGVTTDYADS |
| | | sequence | MKGRFTISRDNAKNTMYLQMNTLKPEDTAIYYC |
| | | | AAKPTWATTNGRPTAYDYWGQGTQVTVSS |
| NO: 21 | AA | D12 CDR1 | GRTFNSFAM |
| NO: 22 | AA | D12 CDR2 | EFVAAIKWNGVTTDY |
| NO: 23 | AA | D12 CDR3 | AAKPTWATTNGRPTAYDY |
| NO: 24 | DNA | Primer CLRDV- | 5′-ACCCTCCAAGGAAC AGAG-3′ |
| | | RdRp-Inner-F | |
| NO: 25 | DNA | Primer CLRDV- | 5′-CGAATAATCTGATYGGGTCAC-3 |
| | | RdRp-Inner-R | |
| NO: 26 | DNA | Primer CLRDV- | 5′-AACGCGCCCAGTCCGCACAAATACC-3′ |
| | | RdRp-Outer-F | |
| NO: 27 | DNA | Primer CLRDV- | 5′-ACCGGGTTTACTGGGGATTGCACGC-3′ |
| | | RdRp-Outer R | |

### DETAILED DESCRIPTION

The present disclosure relates to camelid single-chain antibodies that bind to the cotton leafroll dwarf virus (CLRDV). Using serological or molecular detection assays these camelid single-chain antibodies are useful for detecting CLRDV in plant or aphid samples.

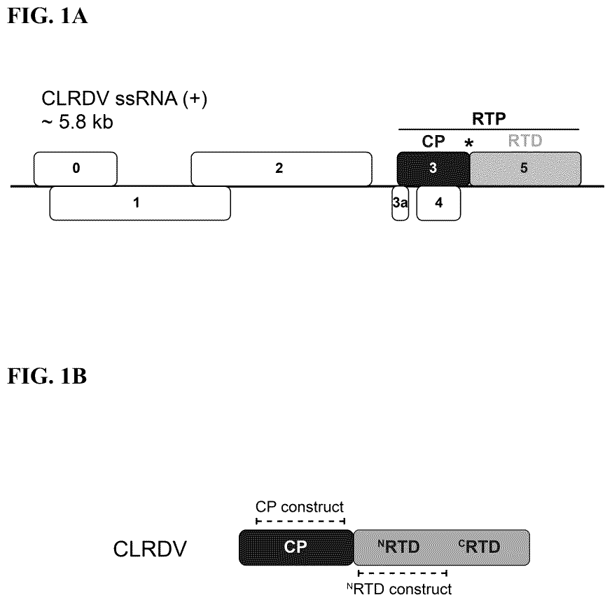

The cotton leafroll dwarf virus (CLRDV) belongs to the genus Polerovirus (family Solemoviridae). The virus has a linear, positive-sense, single-stranded, monopartite RNA genome of approximately 5.8 kb in size, encapsidated in a icosahedral virion of approximately 25-35 nm in diameter. Similar to other poleroviruses, CLRDV is phloem-limited. The cotton aphid (Aphis gossypii Glover) transmits the virus in a persistent, circulative, non-propagative manner. The CLRDV genome is diagramed in FIG. 1A and consists of seven open reading frames (ORFs) partitioned into two regions separated by a 200-nucleotide (nt) intergenic region (IR) that functions in the synthesis of sub-genomic RNAs. The 5′-proximal ORF0 encodes a 28.9 kDa RNA silencing suppressor protein (P0), which is considered to function as an avirulence (AVr) determinant. ORF1 encodes a 70.1 kDa protein (P1) and ORF1-2 encodes the 118.7 kDa viral replication-associated protein P1-P2, which includes the RNA-dependent RNA polymerase (RdRp), which is expressed through a ribosomal frameshift. The 3′-end comprises virus movement and structural genes associated with viral encapsidation (ORF3, black), long-distance movement (ORF3a), cell-to-cell movement (ORF4), and aphid transmission (ORF5, gray). The viral ORF3 encodes the 22.4 kDa coat protein (CP; P3), while ORF4, which is nested within ORF3, encodes the 19.4 kDa movement protein (MP; P4) and is expressed as a frameshift. The ORF3a that encodes the P3a protein (5.2 kDa) is expressed through leaky scanning of the subgenomic RNA. The ORF5 encodes the readthrough domain (RTD) that is expressed through in-frame suppression of the ORF3 stop codon to yield a 77.2 kDa P3-P5 fusion designated together as the readthrough protein (RTP). The CP-RTD is required for aphid transmission and in planta viral accumulation. Finally, the 5′-terminal end is covalently bound to a genome-linked viral protein (VPg), but no poly(A)-tail or tRNA-like structures have been associated with the viral 3′-terminus.

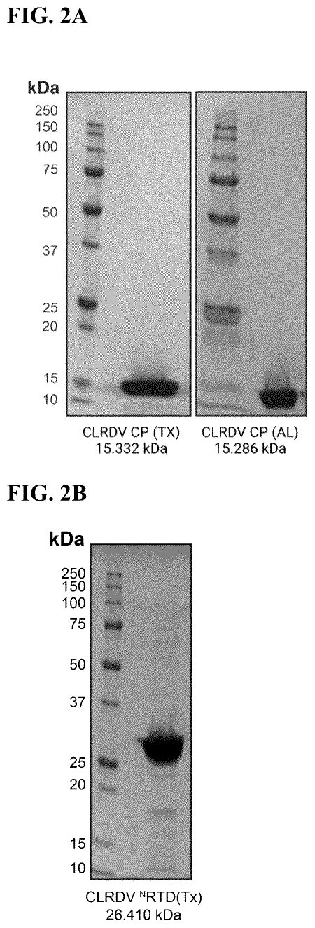

The soluble constructs of the CLRDV CP and the N-terminal segment of the readthrough domain (^{N}RTD) shown in FIG. 1B. Open reading frames encoding the Texas (TX) CLRDV CP, the Alabama (AL) CLRDV CP, and Texas ^{N}RTD constructs were expressed in E. coli and purified according to established protocols for polerovirus structural proteins. As seen in FIG. 2, the TX CLRDV CP had a calculated molecular weight of 15.332 kDa, the AL CLRDV CP had a calculated molecular weight of 15.286 kDa, and the TX CLRDV ^{N}RTD has a calculated molecular weight of 26.410 kDa.

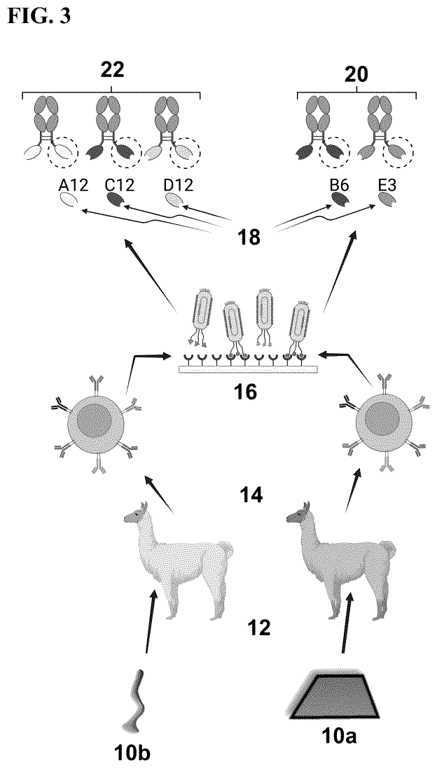

A schematic representation of the preparation of camelid single-chain antibodies specific to CLRDV structural proteins is shown in FIG. 3. The different steps in the preparation arc numbered [10] through [22]. The purified CLRDV CP [10a] and ^{N}RTD [10b] constructs were used to immunize alpacas [12]. Peripheral blood lymphocytes were isolated [14] from the immunized alpacas and, following the generation of a bacteriophage library and panning [16], CLRDV-specific antibodies were identified [18], from which the camelid single-chain antibodies B6 and E3 recognizing the CP [20] and A12, C12, and D12 recognizing the ^{N}RTD [22] were isolated.

The single-chain antibodies described herein were prepared by immunizing alpacas and subsequent screening. This disclosure describes five single-chain antibodies (B6, E3, C12, A12, and D12) derived from immunized alpacas that bind to CLRDV virions recognizing either the CP (B6 and E3) or the ^{N}RTD (C12, A12, and D12).

The sequence of the B6 single-chain antibody is MAQVQLQESGGGLVEAGGSLTLNC TASASIFRGNTMAWYRQAPGEQREFVASITTTGSRNYANSAYGRFTISNDNAKRSVYLH MNSLKPEDTGVYYCNKRFPPQGDWGQGTQVTVSS and is set forth in SEQ ID NO: 8. The complementary determining regions (CDRs) of the B6 camelid single-chain antibody were identified. The sequence of the B6 CDR1 is ASIFRGNTM and is set forth in SEQ ID NO: 9. The sequence of the B6 CDR2 is EFVASITTTGSRNY and is set forth in SEQ ID NO: 10. The sequence of the B6 CDR3 is NKRFPPQGD and is set forth in SEQ ID NO: 11.

The sequence of the E3 antibody is MAQVQLQESGGGLVPPGGSLTLSCTASGFTLG YYAIGWFRQTPGNQRELVASYTSDGHINYKDSVKGRFTISRDGAKNTVWLQMNSLKPE DTAVYYCSFQQWTLVGDDAAQHTDYWGQGTQVTVSS and is set forth in SEQ ID NO: 4. The CDRs of the E3 camelid single-chain antibody were identified. The sequence of the E3 CDR1 is GFTLGYYAI and is set forth in SEQ ID NO: 5. The sequence of the E3 CDR2 is ELVASYTSDGHINY and is set forth in SEQ ID NO: 6. The sequence of the E3 CDR3 is SFQQWTLVGDDAAQHTDY and is set forth in SEQ ID NO: 7.

The sequence of the C12 antibody is MAQVQLQESGGGLAQAGDSLRLSCAASGRTF NSFAMGWFRQAPGKEREFVAAIKWNGVTTDYADSMRGRFTISRDNAKNTMYMQMNT LKPEDTAIYYCAAKPTWATTNGRPTAYDYWGQGTQVTVSS and is set forth in SEQ ID NO: 12. The CDRs of the C12 camelid single-chain antibody were identified. The sequence of the C12 CDR1 is GRTFNSFAM and is set forth in SEQ ID NO: 13. The sequence of the C12 CDR2 is EFVAAIKWNGVTTDY and is set forth in SEQ ID NO: 14. The sequence of the C12 CDR3 is AAKPTWATTNGRPTAYDY and is set forth in SEQ ID NO: 15.

The sequence of the A12 antibody is MAQVQLQESGGGLAQAGDSLRLSCAASGRTF NSFAMGWFRQAPGKEREFVAAINWNGVTTDYADSMKGRFTISRDNAKNTMYLQMNTL KPEDTAIYYCAAKPTWATTNGRPAAYDYWGQGTQVTVSS and is set forth in SEQ ID NO: 16. The CDRs of the A12 camelid single-chain antibody were identified. The sequence of the A12 CDR1 is GRTFNSFAM and is set forth in SEQ ID NO: 17. The sequence of the A12 CDR2 is EFVAAINWNGVTTDY and is set forth in SEQ ID NO: 18. The sequence of the A12 CDR3 is AAKPTWATTNGRPAAYDY and is set forth in SEQ ID NO: 19.

The sequence of the D12 antibody is MAQVQLQESGGGLVQAGDSLRLSCAASGRTF NSFAMGWFRQAPGKEREFVAAIKWNGVTTDYADSMKGRFTISRDNAKNTMYLQMNTL KPEDTAIYYCAAKPTWATTNGRPTAYDYWGQGTQVTVSS and is set forth in SEQ ID NO: 20. The CDRs of the D12 camelid single-chain antibody were identified. The sequence of the D12 CDR1 is GRTFNSFAM and is set forth in SEQ ID NO: 21. The sequence of the D12 CDR2 is EFVAAIKWNGVTTDY and is set forth in SEQ ID NO: 22. The sequence of the D12 CDR3 is AAKPTWATTNGRPTAYDY and is set forth in SEQ ID NO: 23.

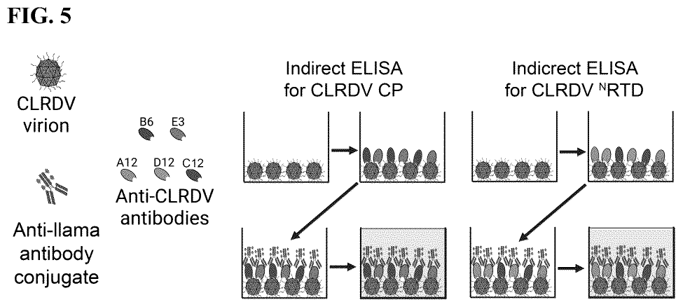

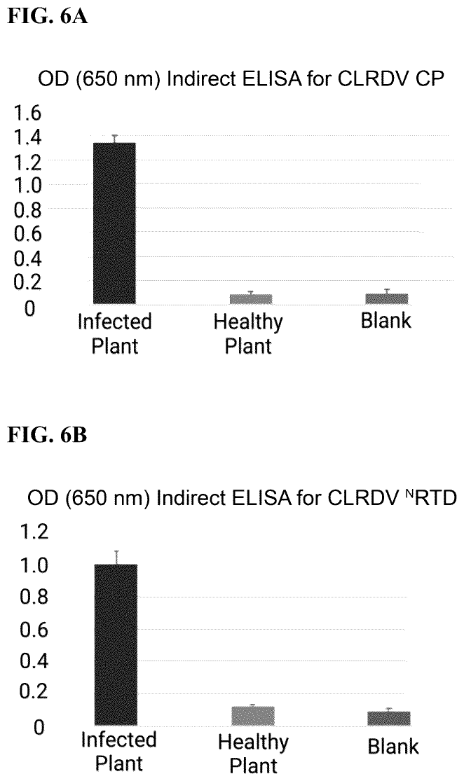

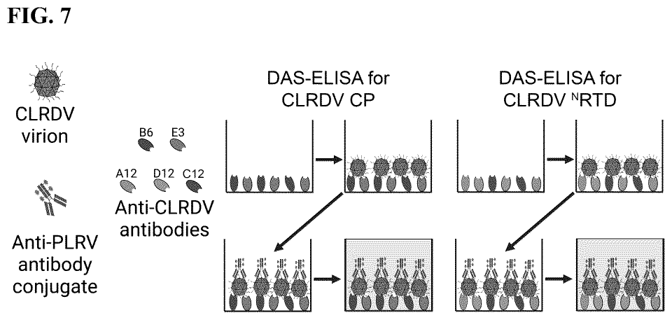

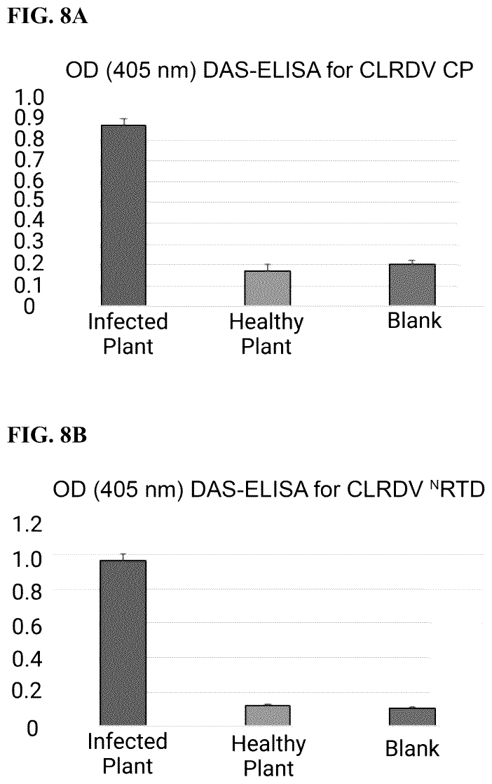

These single-chain antibodies recognize CLRDV from infected cotton plant leaves and from leaf homogenates in a range of diagnostic assays. These assays include immunocapture (IC)-RT-PCR, double antibody sandwich (DAS)-ELISA and indirect ELISA. A schematic diagram of an indirect ELISA assay is shown in FIG. 5, which shows that the polystyrene container is first coated with antigen sample (which may contain CLRDV virions), then the primary antibody (camelid single-chain antibodies recognizing either the CLRDV CP or N′RTD) is added, followed by addition of the secondary antibody conjugate (an anti-llama antibody conjugated with HRP). Positive detection of the substrate is monitored by a colorimetric change in the optical density (OD) at 650 nm. As seen in FIG. 6A, when using the CLRDV CP-specific single-chain antibodies B6 (SEQ ID NO: 8) and E3 (SEQ ID NO: 4) in an indirect ELISA assay, goat anti-llama antibody conjugate, optical density readings at 650 nm were on average 1.3 for infected plants and were below 0.2 for healthy plants and PBS buffer alone. As seen in FIG. 6B, when using the CLRDV ^{N}RTD-specific single-chain antibodies C12 (SEQ ID NO: 12), A12 (SEQ ID NO: 16), and D12 (SEQ ID NO: 20) in an indirect ELISA assay, goat anti-llama antibody conjugate, optical density readings at 650 nm were on average 1.0 for infected plants and were below 0.2 for healthy plants and PBS buffer alone. A schematic diagram for a double-antibody sandwich (DAS) ELISA using either the CLRDV CP-specific single-chain antibodies B6 (SEQ ID NO: 8) and E3 (SEQ ID NO: 4) or ^{N}RTD-specific single-chain antibodies C12 (SEQ ID NO: 12), A12 (SEQ ID NO: 16), and D12 (SEQ ID NO: 20) and a secondary anti-PLRV antibody conjugate is shown in FIG. 7, where the polystyrene container is first coated with antigen sample (which may contain CLRDV virions), then the primary antibody (camelid single-chain antibodies recognizing cither the CLRDV CP or ^{N}RTD) is added followed by addition of the secondary antibody conjugate (anti-PLRV conjugated with AP). Positive detection of the substrate is monitored by a colorimetric change in the OD at 405 nm. As seen in FIG. 8A, when using the CLRDV CP-specific single-chain antibodies B6 (SEQ ID NO: 8) and E3 (SEQ ID NO: 4) in a DAS-ELISA, optical readings at 405 nm averaged 0.85 for CRLDV-infected plants, and less than 0.2 for healthy plants and blank. The results obtained with the ^{N}RTD-specific single-chain antibodies are shown in FIG. 8B.

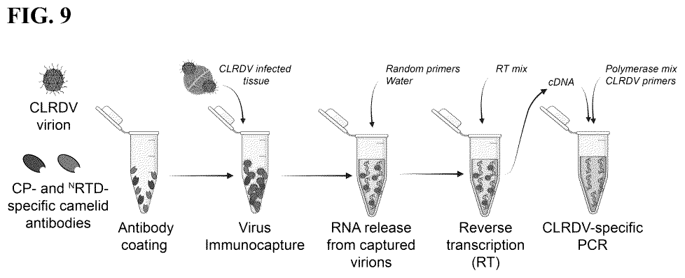

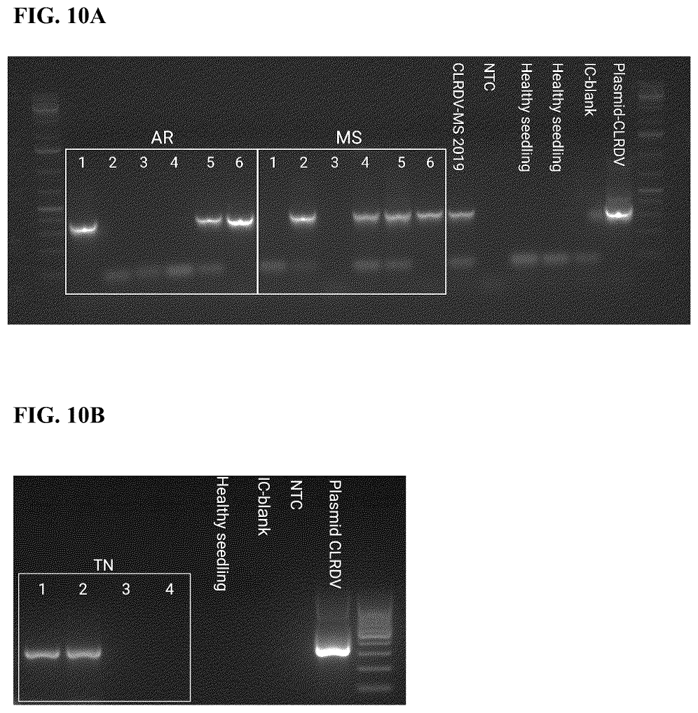

A schematic diagram of an immunocapture (IC)-RT-PCR assay is shown in FIG. 9. In IC-RT-PCR tubes are coated with a mixture of camelid single-chain antibodies followed by immunocapture of CLRDV virions from plant tissue and/or aphids that may be infected. Subsequent heating releases RNA from captured virions, which serves as a substrate for a reverse transcription enzymatic reaction that mix allows for the formation of produces cDNA. The resulting cDNA is then analyzed by polymerase chain reaction (PCR) using CLRDV-specific primers and any amplified products are visualized on an agarose gel under UV illumination. In samples positive for CLRDV, a 350-base pair (bp) product is amplified with a combination of four primers set forth in SEQ ID NO: 24, SEQ ID NO: 25, SEQ ID: NO 26, and SEQ ID NO: 27. IC-RT-PCR allows the detection of RNA present in captured CLRDV virions directly without requiring RNA extraction. As seen in FIG. 10A, the diagnostic assay produced an amplicon of about 350 bp specific for CLRDV in some of the field-collected cotton samples from Mississippi (MS) and Arkansas (AR) that were subjected to immunocapture using the CP-specific single-chain antibodies B6 (SEQ ID NO: 8) and E3 (SEQ ID NO: 4). The amplicon was also obtained in positive controls (CLRDV-MS2019 and Plasmid-CLRDV). No amplicon of a similar size was amplified in the negative controls (non-template control (NTC), healthy seedlings and immunocapture (IC)-blank). These results demonstrated the specificity of the IC-RT-PCR assay. As seen in FIG. 10B, a similar 350 bp amplicon specific for CLRDV was also observed from field-cotton samples from Tennessee (TN) that were subjected to immunocapture using the ^{N}RTD-specific single-chain antibodies C12 (SEQ ID NO: 12), A12 (SEQ ID NO: 16), and D12 (SEQ ID NO: 20).

The single-chain antibodies disclosed herein may be fused to a tag such as yellow fluorescent protein (YFP), green fluorescent protein (GFP), strep tag, FlAsH tag, or a polyhistidine tag (HIS tag).

The present disclosure also provides for plants expressing the single-chain antibodies E3, B6, C12, A12, and D12. In some embodiments, the plants expressing the E3, B6, C12, A12, and D12 single-chain antibodies are transgenic plants. Transgenic plants can be produced as stable transgenic plants, transiently transgenic plants, or modified using symbiont technology.

Unless otherwise explained, all technical and scientific terms used herein have the same meaning as commonly understood by one of ordinary skill in the art to which this disclosure belongs.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used herein, the term “about” is defined as plus or minus ten percent of a recited value. For example, about 1.0 g means 0.9 g to 1.1 g and all values within that range, whether specifically stated or not.

The singular terms “a”, “an”, and “the” include plural referents unless context clearly indicates otherwise. Similarly, the word “or” is intended to include “and” unless the context clearly indicate otherwise.

The term “antibody” refers to an immunoglobulin molecule produced by B lymphoid cells with a specific amino acid sequence. Antibodies are evoked in humans or other animals by a specific antigen (immunogen). Antibodies are characterized by reacting specifically with the target antigen in some demonstrable way, antibody and antigen each being defined in terms of the other. The term includes such variants as monoclonal antibodies, humanized antibodies, single-chain antibodies, and other laboratory-created forms of natural antibodies.

As used herein, the term “single-chain antibody” refers to a camelid monomeric antigen-specific VHH domains in the absence of a constant region. Immunization of Camelidae against targets of interest leads to the in vivo maturation of HCAb and conventional antibody repertoires. Construction of phage-display libraries is performed by cloning of amplified VHH repertoires with minimal modification, thus presenting an authentic picture of in vivo-matured heavy chain repertoire diversity. The potential for direct cloning of VHH repertoires from immunized camelids, the smaller library sizes required to capture the immune VHH repertoire, the stability of the libraries, the feasibility of displaying VHHs on a phage or alternative display formats, and the case of sub-cloning and expression of antigen-specific VHHs are among the major technical advantages of the camelid VHH platform over conventional antibody platforms.

As used herein, the terms “open reading frame” and “ORF” refer to nucleic acid sequences, including both RNA and DNA, that encode genetic information for the synthesis of an RNA, a protein, or any portion of an RNA or protein.

The term “control”, and grammatical variants thereof, is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment. This definition does not refer to internal controls for experiments.

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “CLRDV CP” and synonyms thereof refer to the CLRDV wildtype coat protein. The term “CLRDV ^{N}RTD” and synonyms thereof refer to the wildtype CLRDV N-terminal readthrough domain. The CLRDV TX ORF3-ORF5 has the amino acid sequence MNTVVGRR TINGRRRPRRRNRRRQNQPVVVVQAPRNTQRRRRRRRGGRNRTGGRIPGGPGASSETFV FSKDSLSGSSSGSITFGPSLSDCPAFSNGMLKAYHEYKISMVLLEFISEASSTSSGSISYEV DPHCKLSTLSSTINKFGITKNGRKQFAASFINGQEWHDTSEDQFRILYKGNGSSSIAGSFR VTIRCQFHNPK*VDDGPPPPGPSPPPSPSPPPPVPSRFWGYEGNPQCKILTAENDRNIDSRP LNFVSMYKWEDEKWDKVNLQAGYSRNDRRCMETYFVIPASRGKFHVYLEADGEFVVK HIGGDRDGNWLGNIAYDVSQRGWNIGDYKGCKISNYQSNTVFVAGHPDAEMNGKHFD AARAVEVDWFASFELTCDDEDGAWRIYPPPIQKDSSYNYTVSYGEYTEKYCEWGAVSV SIDEDNSTGTKSRIKPHKGAMMWSDPEKENSEGESEPETSQGKDLKTPDATTLVDFESD DNSSSKSAESIPDYTDTNPWSAVVSSKSDKPFKQEDDRVSTSSRLSGNLRRPGTANPQLR SSLGREKAPEPSESDLDAARIKGLPPPREQPSGFKPTRSISTFNPEPDL VEAWRPGTGPGY SKEDVAAATILAHGSIADGRSMLDKRDQEVLRSRSSWGTGGFIKKIKTSSSDKAEKLAK LSTAERREYELIKNSSGKTQAALFLEQKVMDR*. The top BLAST hit is GenBank ID: QHB18536.1.

The CLRDV AL ORF3-ORF5 has the amino acid sequence MNTVVGRRTINGRRRPR RRNRRRQNQPVVVVQAPRNTQRRRRRRRGGRNRTGGRIPGGPGASSETFVFSKDSLSGS SSGSITFGPSLSDCPAFSNGILKAYHEYKISMVLLEFISEASSTSSGSISYEVDPHCKLSTLS STINKFGITKNGRKQFAASFINGQEWHDTSEDQFRILYKGNGSSSIAGSFKVTIRCQFHNP K*VDDGPPPPGPSPPPSPSPPPPVPSRFWGYEGNPQCKILTAENNRNIDSRPLNFVSMYKW EDEKWDKVNLQAGYSRNDRRCMETYFVIPASRGKFHVYLEADGEFVVKHIGGDRDGN WLGNIAYDVSQRGWNIGDYKGCRISNYQSNAVFVAGHPDAEMDGKHFDAARAVEVD WFASFELTCDDEDGAWRIYPPPIQKDSSYNYTVSYGDYTEKYCEWGAVSVSVDEDNST GTKSRIKPHKGVMMWSHPEKENSEGESESETDQGKDLKTPDATTLVDFDSDDNSSSKSA ESIPDNTDLNPWNAVVSSKSDRPFKQEDDRVSTSSRLSGNLRRPGSGNPQLRSPLGREKA PEPSESDLDAARIKGLPPPREQPPGFKPTRSISTFNPEPDLVEAWRPGTGPGYSKEDVAAA TILAHGSIADGRSMLDKRDQEVLRSRSSWGTGGFLKKMKTSSSDKAEKLAKLSTAERRE YELIKNSSGKTQAALFLEQKVMDR. The top BLAST hit is NCBI Reference Sequence: YP_003915150.1.

The skilled artisan will understand that this disclosure contemplates all DNA and RNA species that encode these proteins, including codon-optimized sequences. This term also refers to mutations of the proteins, or those with added components such as tags, as indicated by a relevant signifier.

The term “polynucleotide” as used herein, refers to a polymer of ribonucleotides or deoxyribonucleotides. Typically, polynucleotide polymers occur in either single- or double-stranded form, but are also known to form structures comprising three or more strands. The term “polynucleotide” includes naturally occurring nucleic acid polymers as well as polymers comprising known nucleic acid analogs or modified backbone residues or linkages, which are synthetic, naturally occurring, and non-naturally occurring. These polynucleotides have similar binding properties as the reference polynucleotide and are metabolized in a manner similar to the reference polynucleotides.

“RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein. For nucleic acids, sizes are given in either kilobases (kb) or base pairs (bp), or nucleotides (nt). Estimates are typically derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., M A. Batzer et al., 1991, “Enhanced Evolutionary PCR Using Oligonucleotides With Inosine at the 3′-Terminus,” Nucleic Acid Res. 19:5081; E. Ohtsuka et al., 1985, “An Alternative Approach to Deoxyoligonucleotides as Hybridization Probes by Insertion of Deoxyinosine at Ambiguous Codon Positions,” J. Biol. Chem. 260:2605-2608; and G M. Rossolini et al., 1994, “Use of Deoxyinosine-Containing Primers vs Degenerate Primers for Polymerase Chain Reaction Based on Ambiguous Sequence Information,” Mol. Cell. Probes 8 (2): 91-98). Because of the degeneracy of nucleic acid codons, one can use various different polynucleotides to encode identical polypeptides.

A “conservative substitution” in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

The term “recombinant polynucleotide” refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seeds (including embryo, endosperm, and seed coat) and fruit (the mature ovary), plant tissue (e.g., vascular tissue, ground tissue, and the like) and cells (e.g., guard cells, egg cells, trichomes and the like).

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized.

Screening and molecular analysis of recombinant organisms can be performed utilizing nucleic acid hybridization techniques. The particular hybridization techniques are not essential to the subject disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989, “Molecular Cloning: A Laboratory Manual,” 2d ed., Cold Spring Harbor Laboratory Press, Plainview, N.Y.) or Ausubel et al. (1995, Current Protocols in Molecular Biology, John Wiley & Sons, NY, N. Y., for further guidance on hybridization conditions.

Screening and molecular analysis of isolated polynucleotides can be performed using Polymerase Chain Reaction (PCR). PCR is a repetitive, enzymatic, primed synthesis of a polynucleotide. This procedure is well known and commonly used by those skilled in this art (see for example, U.S. Pat. Nos. 4,683,195; 4,683,202; and 4,800,159; R K Saiki et al. (1985, “Enzymatic Amplification of, β-Globin Genomic Sequences and Restriction Site Analysis for Diagnosis of Sickle Cell Anemia,” Science 230 (4732): 1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

RT-PCR is that technology by which RNA molecules are converted into their complementary DNA (cDNA) sequences by reverse transcriptases, followed by the amplification of the newly synthesized cDNA by standard PCR procedures.

Recombinant host cells (such as transgenic plant cells or recombinant microbial cells), in the present context, are those which have been genetically modified to contain an isolated polynucleotide, and/or contain one or more genes to produce at least one recombinant protein. Polynucleotides encoding the proteins of the present disclosure can be introduced by any means known to the art to be appropriate for the particular type of cell, including without limitation, transformation, lipofection, electroporation or any other methodology known by those skilled in the art.

Transformation and generation of genetically altered monocotyledonous and dicotyledonous plant cells is well known in the art. Sec, e.g., K. Weising, et al., 1988, “Foreign Genes in Plants: Transfer, Structure, Expression, and Applications,” Ann. Rev. Genet. 22:421-477; U.S. Pat. No. 5,679,558; Agrobacterium Protocols, ed: Gartland, Humana Press Inc. (1995); and M. Wang, et al. (1998, “Improved Vectors for Agrobacterium Tumefaciens-Mediated Transformation of Monocot Plants,” Acta Hort. 461:401-408). The choice of method varies with the type of plant to be transformed, the particular application and/or the desired result. The appropriate transformation technique is readily chosen by the skilled practitioner.

The single-chain antibodies disclosed herein, or fragments thereof, can be utilized in any immunoassay system known in the art including, but not limited to: radioimmunoassays, enzyme-linked immunosorbent assay (ELISA), “sandwich” assays, precipitin reactions, gel diffusion immunodiffusion assays, agglutination assays, fluorescent immunoassays, protein A immunoassays, immunohistochemistry assays, and immunoelectrophoresis. Such assays can be used to detect the presence and/or amounts a target CLRDV in a biological or environmental sample. Single-chain antibodies of the present disclosure can be bound to a solid support in which the immunoassay is to be performed. The solid support can be glass or a polymer, including, but not limited to cellulose, polyacrylamide, nylon, polystyrene, polyvinylchloride or polypropylene. The solid supports can be in the form of tubes, beads, discs microplates, or any other surfaces suitable for conducting an immunoassay.

Single-chain antibodies, or fragments thereof, can be labeled using any of a variety of labels and methods of labeling known to those of skill in the art. Examples of types of labels which can be used in the present invention include, but are not limited to, enzyme labels, radioisotopic labels, non-radioactive isotopic labels, chromogenic labels, fluorescent labels, and chemiluminescent labels (see e.g., Harlow and Lane, Antibodies: A Laboratory Manual [Cold Spring Harbor Laboratory, New York 1988] 555-612).

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Camelid Single-Chain Antibody Preparation

Five different camelid single-chain antibodies were prepared that recognize either the cotton leafroll dwarf virus (CLRDV) coat protein (CP) or the N-terminal segment of the CLRDV read through domain (^{N}RTD).

FIG. 3 depicts a scheme of the preparation of camelid single-chain antibodies using the purified CLRDV CP and ^{N}RTD constructs shown in FIG. 2 as individual antigens for alpaca immunization. CP and ^{N}RTD constructs for the TX and AL CLRDV isolates, as depicted in FIG. 1B, were designed based on previously described crystal structures and purified following established protocols for polerovirus structural proteins as described by M. C. Adams, et al. (2022, “Crystal structure of the potato leafroll virus coat protein and implications for viral assembly,” J. Struct. Biol. 214:107811. doi: 10.1016/j.jsb.2021.107811) and C. J. Schiltz et al. (2022, “Polerovirus N-terminal readthrough domain structures reveal molecular strategies for mitigating virus transmission by aphids.” Nat Commun. 13:6368. doi: 10.1038/s41467-022-33979-2).

Isolation and screening for specific single-chain antibodies was carried out as detailed by K. M. Chow, et al. (2019, “Immunization of Alpacas (Lama pacos) with Protein Antigens and Production of Antigen-specific Single Domain Antibodies,” J. Vis. Exp. (143), e58471. doi: 10.3791/58471). Briefly, alpacas were immunized with ˜400 μg of a mixture of both TX and AL CP or ^{N}RTD preparations that contained 50% (v/v) of adjuvant. Five subsequent weekly boosting injections were provided using ˜200 μg of the same mixture detailed above. Three to five days after the last injection, 50 mL of blood was collected, and lymphocytes purified. A cDNA library was prepared using oligodT primers and RNA extracted from lymphocytes. A bacteriophage library was obtained from the cDNA library by cloning with restriction enzymes into the phage display vector pMES4. Expression of the insert fused to gene III of the filamentous phage was used for production of the phage solution and single-domain antibody panning. After screening, two single-chain antibodies that recognized the CRLDV CP were identified and named B6 (SEQ ID NO: 8) and E3 (SEQ ID NO: 4) and three single-chain antibodies that recognized the CRLDV ^{N}RTD were identified and names C12 (SEQ ID NO: 12), A12 (SEQ ID NO: 16), and D12 (SEQ ID NO: 20), respectively.

The camelid single-chain antibodies were cloned into plasmid pMES4 and expressed in E. coli. The size and purity of the isolated single-chain antibodies was determined by separating on SDS-PAGE. The calculated molecular weight of the B6 antibody (SEQ ID NO: 8) is 13.397 kDa, the calculated molecular weight of the E3 antibody (SEQ ID NO: 4) is 14.440 kDa, the calculated molecular weight of the C12 antibody (SEQ ID NO: 12) is 13.987 kDa, the calculated molecular weight of the A12 antibody (SEQ ID NO: 16) is 13.897 kDa, and the calculated molecular weight of the D12 antibody (SEQ ID NO: 20) is 13.969 kDa.

The information given in this example describes the preparation, purification, and identification of two camelid single-chain antibodies that recognize the CLRDV coat protein and three single-chain antibodies that recognize the CLRDV ^{N}RTD.

### Example 2

Detection of CLRDV with Newly Identified Antibodies

Indirect ELISA and double-antibody sandwich DAS-ELISA were both used to determine their usefulness for the detection of CLRDV in plant and aphid samples using the CP-specific B6 (SEQ ID NO: 8) and E3 (SEQ ID NO: 4) camelid single-chain antibodies and the ^{N}RTD-specific C12 (SEQ ID NO: 12), A12 (SEQ ID NO: 16), and D12 (SEQ ID NO: 20) camelid single-chain antibodies.

For indirect ELISA, plates were coated in triplicates with 100 μL of plant or aphid sample obtained by homogenizing 200-400 mg of tissue in 1 mL of PBS (137 mM sodium chloride, 2.7 mM potassium chloride, 8 mM sodium phosphate dibasic, and 2 mM potassium phosphate monobasic, pH 7.4), and incubated overnight at 4° C. Next; each well was blocked with 100 mg/mL bovine serum albumin (BSA) in PBS for 1 hour at 37° C. Then, 100 μL mixture of either the CP-specific camelid single-chain antibodies B6 (SEQ ID NO: 8) and E3 (SEQ ID NO: 4) (2.5 ng/μL each) or the ^{N}RTD-specific C12 (SEQ ID NO: 12), A12 (SEQ ID NO: 16), and D12 (SEQ ID NO: 20) camelid single-chain antibodies (1.66 ng/μL each) in PBS was added to each well and incubated at 37° C. for 2 hours or overnight at 4° C. Subsequently, 100 μL of goat anti-llama IgG HRP conjugate (Bethyl, Catalog Number: A160-100P) at 1:2,500 dilution in PBS containing 0.4% (w/v) of non-fat dry milk was added to all the wells and incubated for 1 hour at 37° C. After each step, plates were washed three to six times with PBS containing 0.05% Tween 20 (PBS-T). Finally, plates were developed by adding 100 μL of TMB substrate solution (Thermo Fisher Scientific; Waltham, Massachusetts, USA; Catalog number: 34028), and absorbance was read at 650 nm after 1 hour. A schematic diagram of the indirect ELISA assay is shown in FIG. 5. A graph of the resulting optical density measured at 650 nm is shown for the CP-specific single-chain antibodies in FIG. 6A and the ^{N}RTD-specific single-chain antibodies in FIG. 6B. These figures show that in an indirect ELISA, CLRDV-infected plant samples gave OD650 readings of an average of 1.3 when analyzed using a mixture of the CP-specific camelid single-chain antibodies or 1.0 when analyzed using a mixture of the ^{N}RTD-specific camelid single-chain antibodies, while the OD650 readings for healthy plants and blank (PBS buffer only) were less than 0.2 in both cases.

For DAS-ELISA, a 100 μL mixture of either the CP-specific B6 (SEQ ID NO: 8) and E3 (SEQ ID NO: 4) single-chain antibodies (at 2.5 ng/μL each) or B6 (SEQ ID NO: 8) and E3 (SEQ ID NO: 4) (at 5 ng/μL each) or the ^{N}RTD-specific C12 (SEQ ID NO: 12), A12 (SEQ ID NO: 16), and D12 (SEQ ID NO: 20) camelid single-chain antibodies (at 1.66 ng/μL each) in carbonate coating buffer (0.05 M sodium carbonate, 0.05 M sodium bicarbonate, pH 9.6) were coated in triplicates in a plate and incubated for 2 hours at 37° C. Next, 100 μL of plant or aphid sample obtained by homogenizing 200-400 mg of tissue in 1 mL of PBS is added and incubated overnight at 4° C. The next day, 100 μL of anti-PLRV AP conjugate (Agdia; Elkhart, Indiana, USA; Catalog Number: ECA 30002/5000) at 1:200 dilution in PBS was added and incubated for 2 hours at 37° C. After each step, plates were washed eight times with PBS-T. Finally, plates were developed by adding 100 μL of 1 mg/mL of pNPP (Thermo Fisher Scientific, Catalog number: 34045), and absorbance was read at 405 nm after 1 hour. A schematic diagram of the DAS-ELISA assay is shown in FIG. 7. The DAS-ELISA assay comprises four steps to detect CLRDV virions using the camelid single-chain antibodies and a secondary antibody/anti-PLRV conjugated with alkaline phosphatase (AP). The polystyrene container was first coated with the primary antibody (camelid single-chain antibodies recognizing either the CLRDV CP or ^{N}RTD), then antigen sample (which may contain CLRDV virions) was added followed by addition of the secondary antibody conjugate (anti-PLRV conjugated with AP). Positive detection of the substrate was monitored by a colorimetric change in the OD at 405 nm. Graphs of the optical density measured at 405 nm are shown in FIG. 8A for the CP-specific antibodies and FIG. 8B for the ^{N}RTD-specific antibodies. These figures show that in DAS-ELISA, CLRDV-infected plant samples gave OD405 readings of an average 0.85 when analyzed using a mixture of the CP-specific camelid single-chain antibodies, and a reading of 0.90 when analyzed using a mixture of the ^{N}RTD-specific camelid single-chain antibodies, while the OD405 readings for healthy plants and blank were less than 0.2.

For both, indirect (FIG. 6A and FIG. 6B) and DAS-ELISA (FIG. 8A and FIG. 8B), the assays were validated using samples from healthy cotton seedlings, and CLRDV-infected cotton trees that were originally collected from Mississippi in 2019 and maintained since then by fertilization and pruning. A blank sample consisted of PBS buffer only.

The results obtained in this Example show that the presence of CLRDV was definitively identified in infected plants in indirect ELISA or DAS-ELISA assays that use a mixture of either the CP-specific B6 (SEQ ID NO: 8) and E3 (SEQ ID NO: 4) camelid single-chain antibodies or the ^{N}RTD-specific C12 (SEQ ID NO: 12), A12 (SEQ ID NO: 16), and D12 (SEQ ID NO: 20) camelid single-chain antibodies with only background signal seen for negative controls.

### Example 3

### Molecular Diagnostic Approaches

Molecular diagnostic approaches for detection of cotton leafroll dwarf virus (CLRDV) in aphid and cotton tissues using a mixture of either the CP-specific B6 (SEQ ID NO: 8) and E3 (SEQ ID NO: 4) camelid single-chain antibodies or the ^{N}RTD-specific C12 (SEQ ID NO: 12), A12 (SEQ ID NO: 16), and D12 (SEQ ID NO: 20) camelid single-chain antibodies for immunocapture, CLRDV-specific primers targeting a ˜350 bp region in the RNA-dependent RNA polymerase gene of the virus, and PCR assays.

Immunocapture (IC)-RT-PCR assays were adopted by immunocapturing the virions and performing cDNA synthesis in the same tube. An aliquot of the cDNA preparations are then used in RT-PCR assays. Briefly, 100 μL mixtures of either the CP-specific B6 (SEQ ID NO: 8) and E3 (SEQ ID NO: 4) camelid single-chain antibodies or the ^{N}RTD-specific C12 (SEQ ID NO: 12), A12 (SEQ ID NO: 16), and D12 (SEQ ID NO: 20) camelid single-chain antibodies in carbonate coating buffer were added to 0.2 mL tubes and incubated for 2 hours at 37° C. Next, 100 μL of plant or aphid sample obtained by homogenizing 200-400 mg of tissue in 1 mL of PBS was added and incubated overnight at 4° C. After each step, the tubes were washed four times with PBS-T and then rinsed with distilled deionized water. Reverse transcription was performed in the same tube as follows: a mixture of 14 μL DEPC-treated water and 1 μL of random primers (50 ng/μL) was added and tubes were incubated at 95° C. for 5 minutes, which helped the release of RNA in the captured virions. After 2 minutes of ice incubation, 5 μL of reverse transcription mix containing 4 μL of 5× iScript™ select reaction mix, and 1 μl of iScript™ reverse transcriptase (from the iScript™ Select cDNA Synthesis Kit; Bio-Rad; Hercules, California, USA; Catalog number 1708897), was added, and tubes were incubated at 25° C. for 5 minutes followed by 48° C. for 50 minutes and 85° C. for 5 minutes. PCR assays were performed in 20 μL reaction volume as follows: 2 μL of the reverse transcription product was added to 0.2 mL tubes containing 10 μL of 2× Green GoTaq polymerase mix (Promega; Madison, Wisconsin, USA; Catalog number: M7123), 1 μL of CLRDV-RdRp-Inner-F (10 μM, 5′-ACCCTCCAAGGAAC AGAG-3′; set forth in SEQ ID NO: 24), 1 μL of CLRDV-RdRp-Inner-R (10 μM, 5′-CGAATAATCTGATYGGGTCAC-3′; set forth in SEQ ID NO: 25), 1 μL of CLRDV-RdRp-Outer-F (0.1 μM, 5′-AACGCGCCCAGTCCGCACAAATACC-3′; set forth in SEQ ID NO: 26), 1 μL of CLRDV-RdRp-Outer-R (0.1 μM, 5′-ACCGGGTTTACTGGGGATTGCACGC-3′; set forth in SEQ ID NO: 27), and 4 μL of DEPC-treated water. The PCR program consisted of an initial denaturation at 95° C. for 3 minutes followed by 15 cycles of incubation at 94° C. for 30 seconds followed by 62° C. for 30 seconds and 72° C. for 40 seconds. Subsequently, 35 cycles of incubation at 94° C. for 30 seconds followed by 54° C. for 30 seconds and 72° C. for 30 seconds were performed before a final extension cycle at 72° C. for 5 minutes. PCR products were separated in a 2% agarose gel containing GELRED Nucleic Acid Stain at 1× and visualized under UV illumination. The assays were validated using samples from healthy cotton seedlings and a CLRDV-infected cotton tree that was originally collected from Mississippi in 2019 and maintained as detailed above. An artificial positive control consisted in the CLRDV ARG isolate (GenBank accession: GU167940) that was cloned into pJL89 plasmid and used as a positive control for PCR assays. A negative buffer control in the immunocapture step consisting of PBS buffer only, and a non-template water control (NTC) in the PCR assays were also included. The method was further validated using samples collected in 2022 from cotton fields in Arkansas (AR), six samples, and Mississippi (MS), six samples, and Tennessee (TN), four samples.

All the cotton samples originated from plants presenting various viral-like symptoms associated with CLRDV (S. Bag, et al., 2021, “Cotton leafroll dwarf disease: an emerging virus disease on cotton in the US,” Crops Soils 54:18-22; doi: 10.1002/crso.20105). The identity of the CLRDV PCR product was validated using direct Sanger sequencing, and BLAST searches. As seen in FIG. 10A and FIG. 10B, the approximate 350 bp PCR product was obtained in the samples from the artificial positive control (Plasmid-CLRDV), the CLRDV-infected cotton tree (positive control), and three AR samples, four MS samples, and two TN samples that were collected from the field. No band close to the expected product of 350 bp was observed for the negative controls, i.e., buffer negative control (PBS) or the NTC control, as well as the healthy cotton plant. Sanger sequencing confirmed the CLRDV identity of the amplicons corresponding to a partial region of the RNA-dependent RNA polymerase gene of the virus, and all presented nucleotide sequence identities higher than 95% to CLRDV homolog sequence accessions available in the GenBank database.

The results in this Example show that the IC-RT-PCR diagnostic assay using CP-specific and ^{N}RTD-specific camelid single-chain antibodies for immunocapture produced a ˜350 bp amplicon specific for CLRDV in some of the field-collected cotton samples from Mississippi (MS), Arkansas (AR), and Tennessee (TN), as well as the positive controls (CLRDV-MS2019 and Plasmid-CLRDV). No detectable amplicon of this size was visible in the negative controls (non-template control (NTC), healthy seedlings and immunocapture (IC)-blank), demonstrating the specificity of the assay.

We claim:

1. A single-chain antibody or antigen-binding fragment thereof that binds to cotton leafroll dwarf virus (CLRDV).

2. The single-chain antibody or antigen-binding fragment thereof of claim 1, wherein the single-chain antibody or antigen-binding fragment thereof recognizes CLRDV coat protein (CP) or CLRDV n-terminal read-through domain (^{N}RTD).

3. The single-chain antibody or antigen-binding fragment thereof of claim 1, wherein the single-chain antibody or antigen-binding fragment thereof comprises a CDR1, CDR2, and CDR3; wherein, the CDR1 has a sequence as set forth in SEQ ID NO: 5; SEQ ID NO: 9; SEQ ID NO: 13; SEQ ID NO: 17; or SEQ ID NO: 21; the CDR2 has a sequence as set forth in SEQ ID NO: 6; SEQ ID NO: 10; SEQ ID NO: 14; SEQ ID NO: 18; or SEQ ID NO: 22; and the CDR3 has a sequence as set forth in SEQ ID NO: 7; SEQ ID NO: 11; SEQ ID NO: 15; SEQ ID NO: 19; or SEQ ID NO: 23.

4. The single-chain antibody or antigen-binding fragment thereof of claim 3, wherein the CDR1 has a sequence as set forth in SEQ ID NO: 5, the CDR2 has a sequence as set forth in SEQ ID NO: 6, and the CDR3 has a sequence as set forth in SEQ ID NO: 7; or the CDR1 has a sequence as set forth in SEQ ID NO: 9, the CDR2 has a sequence as set forth in SEQ ID NO: 10, and the CDR3 has a sequence as set forth in SEQ ID NO: 11; or the CDR1 has a sequence as set forth in SEQ ID NO: 13, the CDR2 has a sequence as set forth in SEQ ID NO: 14, and the CDR3 has a sequence as set forth in SEQ ID NO: 15; or the CDR1 has a sequence as set forth in SEQ ID NO: 17, the CDR2 has a sequence as set forth in SEQ ID NO: 18, and the CDR3 has a sequence as set forth in SEQ ID NO: 19; or the CDR1 has a sequence as set forth in SEQ ID NO: 21, the CDR2 has a sequence as set forth in SEQ ID NO: 22, and the CDR3 has a sequence as set forth in SEQ ID NO: 23.

5. A vector comprising the single-chain antibody or antigen-binding fragment thereof of claim 1.

6. The vector of claim 5, wherein the vector is an expression vector.

7. A composition comprising the single-chain antibody or antigen-binding fragment thereof of claim 1.

8. A method for determining the presence of CLRDV in a sample, the method comprising:
contacting the sample with at least one single-chain antibody or antigen-binding fragment thereof of claim 1 to produce an antibody/CLRDV complex;
detecting the antibody/CLRDV complex; and
determining that CLRDV is present when the antibody/CLRDV complex is detected.

9. The method of claim 8, wherein the detection of the antibody/CLRDV complex is performed by indirect ELISA or double antibody sandwich ELISA.

10. The method of claim 8, wherein least one single-chain antibody or antigen-binding fragment thereof comprises a heavy chain variable region (VH) comprising CDR1, CDR2, and CDR3; wherein the CDR1 has a sequence as set forth in SEQ ID NO: 5; SEQ ID NO: 9; SEQ ID NO: 13; SEQ ID NO: 17; or SEQ ID NO: 21; the CDR2 has a sequence as set forth in SEQ ID NO: 6; SEQ ID NO: 10; SEQ ID NO: 14; SEQ ID NO: 18; or SEQ ID NO: 22; and the CDR3 has a sequence as set forth in SEQ ID NO: 7; SEQ ID NO: 11; SEQ ID NO: 15; SEQ ID NO: 19; or SEQ ID NO: 23.

11. The method of claim 8, wherein the sample is an aphid sample, a plant sample, or a plant and aphid sample mixture.

12. A method for determining the presence of CLRDV in a sample, the method comprising:
contacting the sample with at least one single-chain antibody or antigen-binding fragment thereof of claim 1 to produce an antibody/CLRDV complex;
releasing RNA from the antibody/CLRDV complex;
using reverse transcriptase to prepare cDNA from the released RNA;
amplifying the cDNA with CLRDV-specific primers; and
determining that CLRDV is present when the CLRDV cDNA is amplified.

13. The method of claim 13, wherein the sample is an aphid sample, a plant sample, or a plant and aphid sample mixture.

14. The method of claim 12, wherein the CLRDV-specific primers have a sequence as set forth in SEQ ID NO: 24; SEQ ID NO: 25; SEQ ID NO: 26; and/or SEQ ID NO: 27.

15. The method of claim 13, wherein the at least one single-chain antibody or antigen-binding fragment thereof comprises a CDR1, CDR2, and CDR3; wherein the CDR1 has a sequence as set forth in SEQ ID NO: 5; SEQ ID NO: 9; SEQ ID NO: 13; SEQ ID NO: 17; or SEQ ID NO: 21; the CDR2 has a sequence as set forth in SEQ ID NO: 6; SEQ ID NO: 10; SEQ ID NO: 14; SEQ ID NO: 18; or SEQ ID NO: 22; and the CDR3 has a sequence as set forth in SEQ ID NO: 7; SEQ ID NO: 11; SEQ ID NO: 15; SEQ ID NO: 19; or SEQ ID NO: 23.

16. A plant or plant part comprising a single-chain antibody or antigen-binding fragment thereof, which antibody or antigen-binding fragment thereof comprises a CDR1, a CDR2, and a CDR3; wherein the CDR1 has a sequence as set forth in SEQ ID NO: 5, SEQ ID NO: 9, SEQ ID NO: 13, SEQ ID NO: 17, or SEQ ID NO: 21; the CDR2 has a sequence as set forth in SEQ ID NO: 6, SEQ ID NO: 10, SEQ ID NO: 14, SEQ ID NO: 18, or SEQ ID NO: 22; and the CDR3 has a sequence as set forth in SEQ ID NO: 7, SEQ ID NO: 11, SEQ ID NO: 15, SEQ ID NO: 19, or SEQ ID NO: 23.
